(19) United States
(12) Patent Application Publication
(10) Pub. No.: US 2015/0368192 A1
(43) Pub. Date: Dec. 24, 2015

(54) A METHOD OF SYNTHESIZING CREATINE DERIVATIVES

(71) Applicant: UNIVERSITA' DEGLI STUDI DI GENOVA, Genova (IT)

(72) Inventors: Enrico Millo, Genova (IT); Maurizio Balestrino, Genova (IT); Gianluca Damonte, Arenzano (Genova) (IT); Patrizia Garbati, Varazze (Savona) (IT); Enrico Adriano, Genova (IT); Annalisa Salis, Quiliano (Savona) (IT)

(21) Appl. No.: 14/650,157

(22) PCT Filed: Nov. 18, 2013
(86) PCT No.: PCT/IT2013/000323

§ 371 Date: Jun. 5, 2015

(30) Foreign Application Priority Data
Dec. 18, 2012 (IT) TO2012A001098

(51) Int. Cl. C07C 277/08; C07F 9/09

(52) U.S. Cl. CPC C07C 277/08; C07F 9/09

(57) ABSTRACT

A method of synthesizing (Boc)₂-creatine derivatives of formula (III) which comprises a first step in which a sarcosine ester is reacted with a guanylating agent comprising two nitrogen atoms each protected with a t-butoxycarbonyl (t-Boc) group to form a (Boc)₂-creatine ester, and a second step in which the (Boc)₂-creatine ester is subjected basic hydrolysis to form (Boc)₂-creatine of formula (III) is described. The (Boc)₂-creatine so obtained can be used in methods of synthesizing creatine and phosphocreatine derivatives in which the free carboxyl group of the creatine is conjugated with a desired molecule.

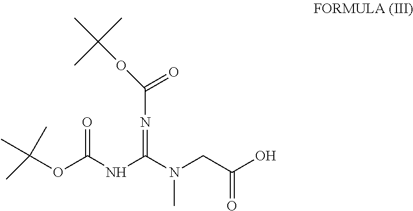 [SMILES: CN(CC(=O)O)C(=NC(=O)OC(C)(C)C)NC(=O)OC(C)(C)C]

This invention relates to a method of synthesizing creatine derivatives.

Intracellular ATP levels are maintained constant through the reversible phosphorylation of creatine to phosphocreatine, performed by the enzyme creatine kinase. Phosphocreatine is in fact capable of donating a phosphate group to ADP, restoring ATP levels. Creatine thus has a central part to play in cell energy metabolism. Its action is of great importance in all cell types, mainly in muscular tissue and in the brain.

As is well known, creatine transfers a phosphate group to ATP using the enzyme creatine kinase according to the following reaction:

Cr+ATP [symbol] PCr+ADP+H⁺

Cr=Creatine

PCr=Phosphocreatine

ATP=Adenosine triphosphate

ADP=Adenosine diphosphate

Creatine is synthesized in the kidneys, liver, pancreas and brain, or it is obtained from food sources such as fresh meat and fish. It is transported through the blood and enters the cells of tissues, particularly those having a high energy demand, such as in particular muscle and brain cells, using its own specific transporter (CrT). The transporter is required so that the creatine can cross the blood-brain barrier.

Creatine deficiency syndromes represent a group of diseases caused by mutations in the genes for arginine glycine amidinotransferase (AGAT, EC 2.1.4.1) and guanidinoacetate methyltransferase (GAMT, EC 2.1.1.2), two enzymes which are required for the synthesis of creatine, and the SLC6A8 gene which codes for the specific creatine transporter.

Patients affected by these syndromes manifest severe neurological symptoms in early infancy, which typically include mental retardation and epileptic crises of variable severity, but there are often other symptoms such as delayed language development, movement disorders and behavioural disorders, including autism, hyperactivity and self-harming.

Creatine transporter deficiency is currently an incurable disease and one possible solution might lie in the administration of creatine in a form capable of crossing biological membranes without the help of the specific creatine transporter, which is absent in these patients. Thus the administration of creatine would be of great benefit, including for other diseases characterised by creatine deficiency, which also include ischaemic jaundice in addition to the abovementioned AGAT and GAMT deficiency syndromes.

However creatine is a polar molecule which is not readily able to cross biological membranes. In order to overcome this disadvantage it is therefore necessary to have creatine derivatives which increase its lipophilic nature and therefore make it suitable for crossing biological membranes, preferably without the help of its specific transporter. An alternative strategy comprises binding it to other molecules which can perform the function of carrier and therefore carry it across biological membranes by means of other transporters.

One technical problem associated with the synthesis of creatine derivatives, however, lies in the fact that it is not very reactive with other molecules, because of its low solubility in water and organic solvents.

US 2009/0297685 describes a method of synthesizing imino-sugars bound to creatine which in a first step provides for the synthesis of creatine protected by t-butoxycarbonyl (hereafter indicated as “(Boc)₂-creatine”) on the two nitrogen atoms of the guanidine group; this form is in fact more stable and more reactive than unprotected creatine. (Boc)₂-creatine also has the advantage that the carboxyl group is unprotected and therefore free to react with other molecules to form the desired derivative. In creatine derivatives which are suitable for the treatment of creating deficiency syndromes it is in fact necessary that the bond with the molecule of interest should be a covalent bond which does not involve the guanidine group of the creatine, which must be left free to interact with the enzyme creatine kinase.

According to the teaching in US 2009/0297685, (Boc)₂-creatine is synthesized in the aqueous phase by causing creatine to react with N,N-bis(t-butoxycarbonyl)anhydride. This method however has the disadvantage that it offers low yields because of the instability and low solubility of the creatine.

The object of this invention is therefore to provide a method of synthesizing (Boc)₂-creatine and subsequently creatine derivatives which overcomes the problems in the prior art.

This object is accomplished through a method of synthesizing (Boc)₂-creatine as defined in the characterising part of claim 1.

(Boc)₂-creatine synthesized by the method according to the invention has the structural formula illustrated below as formula (III):

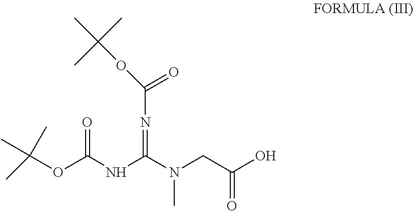 [SMILES: CN(CC(=O)O)C(=NC(=O)OC(C)(C)C)NC(=O)OC(C)(C)C]

The first step in the method according to the invention provides for the use of a sarcosine ester of formula (I) as a precursor which is converted into (Boc)₂-creatine ester of formula (II) according to a simple procedure. The ester of formula (II) is in fact obtained through using a guanylating agent protected with t-Boc on both nitrogen atoms, which allows it to be synthesized directly.

The sarcosine ester of formula (I) used as a precursor in the first step of the method according to the invention has the structural formula illustrated below:

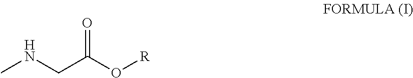

in which R is a linear or branched saturated or unsaturated alkyl or aryl group having from 1 to 8 carbon atoms. In a preferred embodiment, R is a linear alkyl group having 1 to 8 carbon atoms; even more preferably, R is ethyl and formula (I) therefore represents the ethyl ester of sarcosine.

The (Boc)₂-creatine ester of formula (II) has the following structural formula:

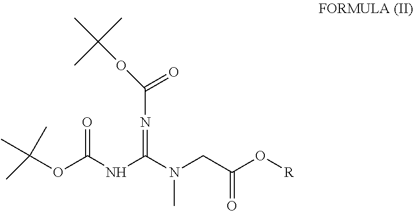

in which R is as defined for formula (I).

In the next step of the method, the (Boc)₂-creatine ester of formula (II) is subjected to basic hydrolysis to form the (Boc)₂-creatine of formula (III).

The method according to the invention advantageously makes it possible to achieve high yields and optimum purity for the final (Boc)₂-creatine product. In a preferred embodiment 1,3-bis(t-butoxycarbonyl)-2-methyl-2-thiopseudourea (CAS 107819-90-9) or N,N-bis(t-butoxycarbonyl) 1-guanyl pyrazole (CAS 152120-54-2) is used as the guanylating agent. The yields obtained with these two guanylating agents are substantially similar.

In a second aspect of the invention, the (Boc)₂-creatine of formula (III) obtained by the method described above is subsequently used to synthesize a creatine derivative through conjugation using conventional procedures with a molecule comprising a functional group capable of reacting with the free carboxyl group of the creatine, thereby obtaining a (Boc)₂-creatine derivative.

Non-limiting examples of molecules comprising a functional group capable of reacting with the free carboxyl group of (Boc)₂-creatine of formula (III) are amino acids and their esters, amines, alcohols, thiols, lipids, vitamins and carbohydrates.

Finally, if so desired, the two t-Boc groups may be easily removed from the (Boc)₂-creatine derivative by treatment in an acid environment in order to obtain a creatine derivative which optionally may in turn be used as a precursor for the synthesis of further derivatives in which the guanidine group of the creatine is modified by bonding to any molecule comprising a functional group capable of reacting with the guanidine group of the creatine. Preferred derivatives modified on the guanidine group of the creatine are illustrated by the following structural formula (IV):

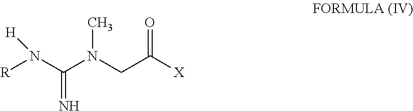

in which X is a residue of a molecule as defined in the appended claims and R is selected from the group comprising —OH, —PO(R₁)(R₂), —COR₃ and —SO₂R₄, in which R₁ and R₂ are independently selected from the group comprising hydrogen, hydroxyl and —OR₅, and in which R₃, R₄ and R₅ are independently selected from the group comprising linear or branched C1-C16 alkyl and heteroalkyl groups, cycloalkyl groups and C3-C8 heterocycloalkyls, which may be substituted, and aryl and heteroaryl groups which may be substituted.

Particularly preferred creatine derivatives included in formula (IV) are phosphocreatine derivatives in which —R is —PO(OH)(OH), which are obtained by causing the precursor to react with a phosphorylating agent.

The use of t-Boc as a group protecting the guanidine group according to the method of the invention is particularly advantageous for the synthesis of creatine derivatives. The present inventors have in fact tried using other protected groups described in the literature, and have experimented with different methods to protect the guanidine group, such as the insertion of the p-toluenesulfonyl group, the insertion of a trityl (triphenylmethyl) group and the insertion of the Pbf (2,2,4,6,7-pentamethyldihydrobenzofuran-5-sulfonyl) group without however achieving satisfactory results, in that the attempts resulted in degradation of the product and/or yields which were too low.

The examples below are provided for merely illustrative purposes and do not limit the scope of the invention as defined by the appended claims.

### EXAMPLE 1

### Synthesis of (Boc)₂-Creatine

Method a Using 1,3-Bis(t-Butoxycarbonyl)-2-Methyl-2-Thiopseudourea as the Guanylating Agent (Reaction Scheme 1)

1.1 equivalents of HgCl₂ were added to a solution of sarcosine ethyl ester (1.2 equivalents), 1,3-bis(t-butoxycarbonyl)-2-methyl-2-thiopseudourea (1 equivalent) and triethylamine (3 equivalents) in anhydrous N,N dimethylformamide. The suspension was kept stirring at ambient temperature until completion of the reaction, which was monitored using thin layer chromatography (TLC). Indicatively, depending upon the quantities used, reaction times varied from 18 to 24 hours. On completion the reaction mixture was taken up in ether with the formation of an abundant white precipitate. This precipitate was filtered off under vacuum. The ethereal solution obtained was washed twice with deionised water and subsequently a further 2 times with a solution of NaCl (0.1M). The ether phase was evaporated to minimum volume and subsequently lyophilised. A solution of acetonitrile and 1N NaOH in a 1:1 ratio was added to the product so obtained, while stirring, in order to hydrolyse the ethyl group. This reaction was also monitored using TLC. On completion of the reaction the pH of the solution was raised to 6 using 1N HCl. The compound was then centrifuged to remove any precipitate. The supernatant was lyophilised, yielding a white powder. The structure of the molecule was verified by mass spectrometric analysis, which confirmed the expected molecular weight.

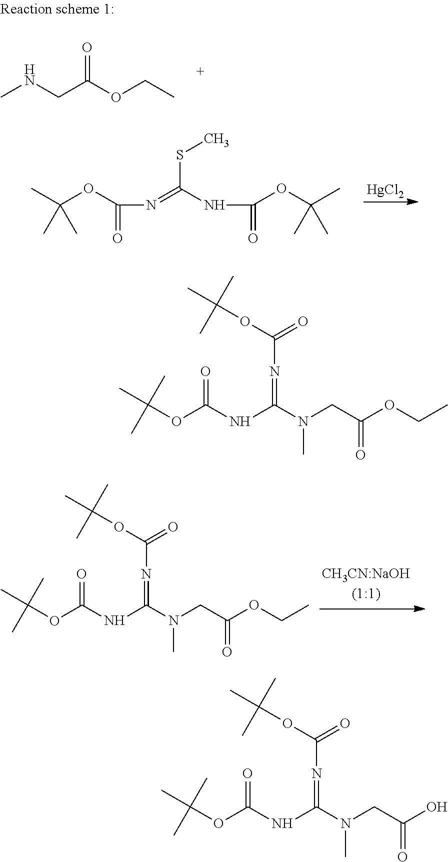

### EXAMPLE 2

### Synthesis of (Boc)₂-Creatine

### Method B Using N,N-Bis(t-Butoxycarbonyl)-1-Guanyl Pyrazole as Guanylating Agent (Reaction Scheme 2)

A solution of sarcosine ethyl ester (1.2 equivalents), N,N-bis(t-butoxycarbonyl)-1-guanyl pyrazole (1 equivalent) and triethylamine (3 equivalents) in anhydrous N,N-dimethylformamide was maintained at ambient temperature with stirring until the reaction was complete. The reaction was monitored using TLC. Indicatively, depending upon the quantities used, reaction times varied from 18 to 24 hours. On completion the reaction mixture was taken up in ether and washed twice with an equivalent quantity of deionised water. The ether phase was evaporated to minimum volume and subsequently lyophilised. A solution of acetonitrile and 1N NaOH in a 1:1 ratio was added to the product so obtained, with stirring, in order to hydrolyse the ethyl group. On completion of the reaction the pH of the solution was increased to 6 using 1N HCl. The compound was then centrifuged to remove any precipitate. The supernatant was lyophilised to yield a white powder. The structure of the molecule was checked by mass spectrometric analysis, confirming the molecular weight expected.

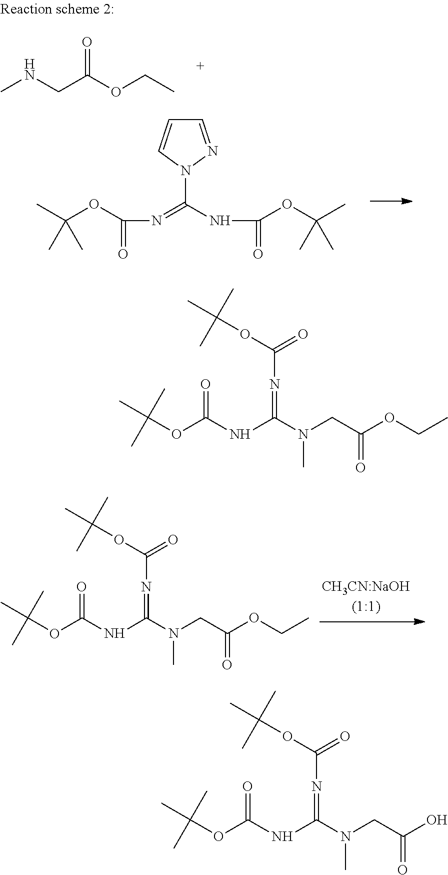

### EXAMPLE 3

### Synthesis of (Boc)₂-Creatine Bound to an Esterified Amino Acid

One equivalent of (Boc)₂-creatine was dissolved in anhydrous N,N-diethylformamide. 1 equivalent of isobutyl chloroformate and 1 equivalent of N-methylmorpholine was added to this solution, kept with stirring at a temperature of 0° C. After 10 minutes the reaction was brought to ambient temperature and protected from the light. Esterified amino acid previously prepared by stirring the amino acid ester present in the form of hydrochloride (1.5 equivalents) with triethylamine (3 equivalents) in anhydrous N,N-dimethylformamide for 30 minutes was added to this solution. The mixture obtained was then centrifuged and the supernatant added to the mixture containing activated (Boc)₂-creatine. The compound was kept stirring at ambient temperature for between 24 and 48 hours, depending upon the amino acid, the progress of the reaction being monitored using TLC. On completion of the reaction the synthesis mixture was centrifuged and the supernatant obtained was lyophilised. This product was taken up in ether or ethyl acetate, depending upon the polarity of the amino acid used, and washed with deionised water. The organic phase was then evaporated to minimum volume. The creatine derivative so obtained was purified by reverse phase HPLC (high performance liquid chromatography). A solution of dichloromethane and trifluoroacetic acid in a 1:1 ratio was added to the final product, brought to a temperature of 0° C. (Reaction scheme 3). On completion of the reaction, which was monitored by TLC, the compound was added dropwise to cold ether, yielding a white precipitate. The precipitate was separated out by centrifuging and dried to a powder by lyophilisation.

The structure of the derivatives obtained was confirmed by mass spectrometric analysis, confirming the expected molecular weight.

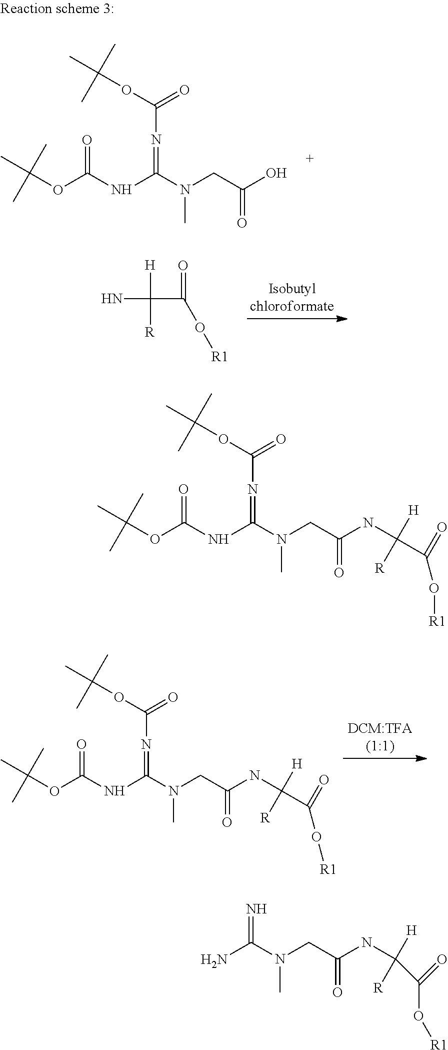

### EXAMPLE 4

### Synthesis of Creatine Amides

One equivalent of (Boc)₂-creatine was dissolved in anhydrous N,N-dimethylformamide. 1 equivalent of isobutyl chloroformate and 1 equivalent of N-methylmorpholine were added to this solution, which had been brought to a temperature of 0° C. After 10 minutes, the reaction was brought to ambient temperature and protected from the light. 1.5 equivalents of amine were added to this solution. The mixture was kept stirring at ambient temperature for between 24 and 48 hours, depending upon the amine used, monitoring the reaction using TLC. On completion of the reaction, the suspension was centrifuged and the supernatant was lyophilised. The lyophilised compound was then taken up in ether and washed with deionised water. The organic phase was then evaporated to minimum volume and finally lyophilised. The product so obtained was purified by means of reverse phase HPLC. A solution of dichloromethane and trifluoroacetic acid in a 1:1 ratio was added to the final product, brought to a temperature of 0° C. (Reaction scheme 3). On completion of the reaction, which was monitored using TLC, the product obtained was added dropwise to cold ether, yielding a precipitate. The precipitate was separated out by centrifuging and dried to a powder by lyophilisation.

The following derivatives were prepared by this method: creatine-piperidine, creatine-paratoluidine, creatine-morpholine, creatine-diethylamine. The structure of the derivatives prepared was confirmed by mass spectrometric analysis, confirming the expected molecular weights.

Of course the same procedure may be used for the synthesis of any creatine derivative which can be obtained through reaction with a molecule having at least one amine group.

### EXAMPLE 5

### Synthesis of Phosphocreatine Derivatives with a Protected Phosphate Group

The creatine derivative obtained by the synthesis according to examples 3 and 4 (1 equivalent) was dissolved in anhydrous pyridine and dichloromethane (1:5). DMAP (1 equivalent) was added to the mixture, which was kept stirring at ambient temperature. A solution of diphenylchlorophosphate (1 equivalent) in anhydrous pyridine and dichloromethane (1:5) was subsequently added dropwise to the mixture.

The reaction was monitored using TLC (hexane: ethyl acetate, 1:1). On completion of the reaction the suspension was centrifuged and the supernatant was evaporated to minimum volume. The compound was then taken up in diethylether and washed several times with deionised water. The organic phase was then evaporated to minimum volume and finally lyophilised. The product so obtained was purified by reverse phase HPLC.

### EXAMPLE 6

### Synthesis of Phosphocreatine Derivatives with a Protected Phosphate Group

The creatine derivative obtained through the synthesis according to examples 3 and 4 (1 equivalent) was dissolved in anhydrous tetrahydrofuran in the presence of triethylamine (1 equivalent). The reaction was cooled to 10° C. and 1 equivalent of diphenylchlorophosphate dissolved in anhydrous tetrahydrofuran was added dropwise to it. After addition the reaction temperature was raised to 40° C. until the product formed, monitoring using TLC (ethyl acetate: hexane, 6:4). On completion of the reaction the mixture was evaporated to minimum volume. The compound was then taken up in diethylether and washed several time with deionised water. The organic phase was then evaporated to minimum volume and finally lyophilised.

The product was subsequently purified using HPLC and lyophilised.

### EXAMPLE 7

### Synthesis of Phosphocreatine Derivatives with an Unprotected Phosphate Group

The product obtained in examples 5 and 6 was dissolved in tetrahydrofuran and water (5:2) in the presence of NaOH (1 equivalent). The mixture was stirred at a temperature of 40° C. and monitored using TLC (ethyl acetate and methanol, 9:1) until completion. The product was then purified using HPLC and lyophilised.

1. A method of synthesizing (Boc)₂-creatine of formula (III), comprising the steps of:
(i) reacting a sarcosine ester of formula (I) 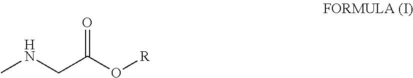
wherein R is a linear or branched, saturated or unsaturated alkyl or aryl group having 1 to 8 carbon atoms,
with a guanylating agent comprising two nitrogen atoms each protected with a t-butoxycarbonyl group (t-Boc),
to form a (Boc)₂-creatine ester of formula (II) 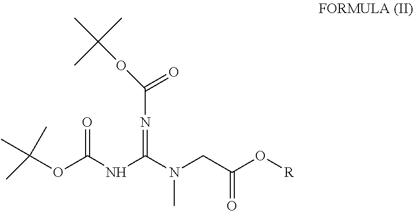
wherein R is a linear or branched, saturated or unsaturated alkyl or aryl group having 1 to 8 carbon atoms; and
(ii) subjecting the (Boc)₂-creatine ester of formula (II) to basic hydrolysis, to form (Boc)₂-creatine of formula (III)

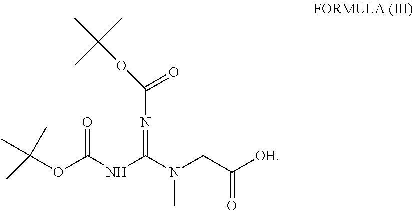 [SMILES: CN(CC(=O)O)C(=NC(=O)OC(C)(C)C)NC(=O)OC(C)(C)C]

2. The method according to claim 1, wherein R is a linear alkyl group.

3. The method according to claim 2, wherein R is a linear saturated alkyl group.

4. The method according to claim 3, wherein R is ethyl.

5. The method according to claim 1, wherein the guanylating agent is 1,3-bis(t-butoxycarbonyl)-2-methyl-2-thiopseudourea or N,N-bis(t-butoxycarbonyl)-1-guanyl-pyrazole.

6. A method of synthesizing a creatine derivative, comprising of synthesizing (Boc)₂-creatine of formula (III) 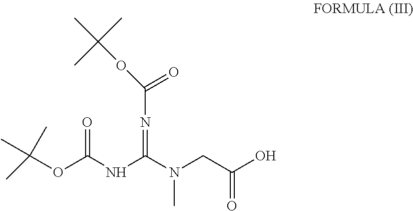 [SMILES: CN(CC(=O)O)C(=NC(=O)OC(C)(C)C)NC(=O)OC(C)(C)C]
by a method according to claim 1, and conjugating the (Boc)₂-creatine of formula (III) with a molecule comprising a functional group capable of reacting with the free carboxyl group of (Boc)₂-creatine of formula (III), thereby obtaining a derivative of (Boc)₂-creatine.

7. The method according to claim 6, wherein the molecule comprising a functional group capable of reacting with the free carboxyl group of the (Boc)₂-creatine of formula (III) is selected from the group consisting of amino acids and their esters, amines, alcohols, thiols, lipids, vitamins and carbohydrates.

8. The method according to claim 5, comprising the further step of removing the t-butoxycarbonyl groups from the (Boc)₂-creatine derivative through treatment in an acid environment, thereby obtaining a creatine derivative.

9. The method according to claim 8, comprising the further step of reacting the creatine derivative with a molecule comprising one or more functional groups capable of reacting with the guanidine group of the creatine derivative, thus obtaining a creatine derivative which is modified on the guanidine group.

10. The method according to claim 9, wherein the creatine derivative modified on the guanidine group is represented by structural formula (IV): 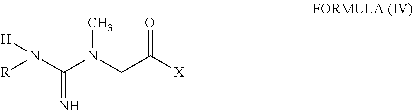
wherein:
X is a functional group capable of reacting with the free carboxyl of the compound of formula (IV);
R is selected from the group consisting of —OH, —PO(R₁)(R₂), —COR₃ and —SO₂R₄; R₁ and R₂ are independently selected from the group consisting of hydrogen, hydroxyl and —OR₅; and
R₃, R₄ and R₅ are independently selected from the group consisting of linear or branched C1-C16 alkyl and heteroalkyl groups, cycloalkyl groups and C3-C8 heterocycloalkyl groups, optionally substituted, and aryl and heteroaryl groups, optionally substituted.

11. The method according to claim 11, wherein R is —PO(R₁)(R₂) and R₁ and R₂ are both hydroxyl.

12. The method according to claim 10, wherein X is selected from the group consisting of amino acids and their esters, amines, alcohols, thiols, lipids, vitamins and carbohydrates.
